(19) United States
(12) Patent Application Publication
(10) Pub. No.: US 2014/0349977 A1
(43) Pub. Date: Nov. 27, 2014

(54) EPIGENETIC MARKERS FOR DETECTION OF AUTISM SPECTRUM DISORDERS

(71) Applicant: ZYMO RESEARCH CORPORATION, Irvine, CA (US)

(72) Inventors: Xueguang Sun, Tustin, CA (US); Xiyu Jia, Irvine, CA (US)

(73) Assignee: ZYMO RESEARCH CORPORATION, Irvine, CA (US)

(21) Appl. No.: 14/350,979

(22) PCT Filed: Oct. 12, 2012
(86) PCT No.: PCT/US12/59916

§ 371 Date: Apr. 10, 2014

Related U.S. Application Data
(60) Provisional application No. 61/547,383, filed on Oct. 14, 2011.
(60) Provisional application No. 61/609,499, filed on Mar. 12, 2012.

(51) Int. Cl. C12Q 1/68

(52) U.S. Cl. CPC C12Q 1/68
USPC 514/152; 435/6.11; 506/9

(57) ABSTRACT

Methods for determining if a patient has, or is at risk of having, and autism spectrum disorder by detecting epigenetic changes in the genome of the patient. For example, a method can comprise determining the methylation status of one or more genes in a blood sample.

This application claims the benefit of U.S. Provisional Patent Application Nos. 61/547,383, filed Oct. 14, 2011, and 61/609,499, filed Mar. 12, 2012, each of which are incorporated herein by reference in their entirety.

### BACKGROUND OF THE INVENTION

1. Field of the Invention

The present invention relates generally to the field of molecular biology, epigenetics and predictive medicine. More particularly, it concerns methods for detecting autism spectrum disorders by determining epigenetic modifications in the genome.

2. Description of Related Art

Autism spectrum disorders (ASD) constitute a group of related childhood neurodevelopmental disorders characterized by deficits in the development of language skills and social relationships, patterns of repetitive behaviors, restricted interests and a strong desire to maintain “sameness” of environment (Folstein et al., 2001). The disorders are typically apparent by three years of age and are more prevalent in males by a ratio of nearly 4:1. Today the overall prevalence of ASD diagnosis is approaching 0.6% of live births, thus the disorders pose a significant medical and economic concern.

Classic autism represents the severe end of autism spectrum disorders, which also include Asperger syndrome, pervasive developmental disorder not otherwise specified (also known as atypical autism), Rett syndrome and childhood disintegrative disorder. Evidence from twin studies indicates that ASD has a genetic component (Bailey et al., 1995). Nonetheless, the molecular basis of ASD remains elusive and ASD diagnosis is restricted to subjective behavioral observation. There is currently no biomarker assay, genetic or otherwise, that can be used to diagnose or assess risk for ASD.

### SUMMARY OF THE INVENTION

In a first embodiment a method is provided for detecting the presence, severity, or an increased risk of, an autism spectrum disorder (ASD) in a patient. In some aspects, a method of the embodiments comprises determining a methylation status in one or more genes in a patient sample wherein an increased or decreased level of methylation in one or more of the genes relative to a reference level indicates that the patient has or is at risk of having ASD or severe ASD. In further aspects, a method comprises (i) determining a methylation status in one or more genes in a patient sample; and (ii) identifying the presence of, the severity of or an increased risk of, ASD in the patient based on an increased or decreased level of methylation in one or more of the genes relative to a reference level. For example, the method can be used to detect the presence, severity or risk of developing classic autism, Asperger syndrome, atypical autism, Rett syndrome or childhood disintegrative disorder. In certain aspects, a method of the embodiments is further defined as an in vitro method.

Some aspects of the embodiments concern determining a methylation status in one or more genes in a patient sample. For example, the genes can be selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2, and POM121 wherein an increased level of methylation in one or more of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, POM121, NLGN2, and/or a decreased level of methylation in one or more of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, or INGX relative to a reference level indicates that the patient has or is at risk of having an autism spectrum disorder. As used herein the term “gene” refers to DNA region that encodes a protein or RNA as well as regions controlling expression of the RNA or protein (e.g., promoter/enhancer regions, insulator sequences, termination sequences and introns). Thus, in some aspects, a methylation status can be determined for 1, 2, 3, 4, 5, 6, 7, 8, 9, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or all 99 of the indicated genes. As used herein “determining a methylation status” for an indicated gene means determining whether one of more position in the DNA of the gene is methylated. Thus, in certain aspects, determining a methylation status for a gene comprises determining the methylation status at least 1, 2, 3, 4, 5, 6, 7, 8, 9, 10, 15, 20, 25, 30, 35, 40, 45, 50 or more sites of potential DNA methylation. In certain aspects, a methylation status is determined for a promoter/enhancer region of a gene, such as a portion of the gene indicated in Table 1.

In some aspects, for example, a methylation status is determined for one or more genes selected from the group consisting of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, NLGN2 and POM121 (e.g. SPIN3, ANKRD13C, GHSR, BDNF, CMYA5, MCRS1, MIR548H4, NARG2, CES1, SNCA, ITGA4 and NLGN2) wherein an increased level of methylation in one or more of the genes relative to a reference level indicates that the patient has or is at risk of having an autism spectrum disorder. In certain cases, a methylation status is determined for one, two or all three of the genes selected from CES1, SNCA and ITGA4 wherein an increased level of methylation in one or more of the genes relative to a reference level indicates that the patient has or is at risk of having an autism spectrum disorder.

In still further aspects, a methylation status is determined for one or more genes selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, INGX, and S100A13 (e.g., NGDN, OTOF_{—}1, MTBP, MRPL13, HOXB6, LCK, ANAPC7, SEC1, ABCE1, ANAPC10, CD1D, ALKBH6, S100A13 and IGNX) wherein a decreased level of methylation in one or more of the genes relative to a reference level indicates that the patient has or is at risk of having an autism spectrum disorder. In some cases, a methylation status is determined for one, two, three or more genes selected from the group consisting of MTBP, SEC1, ABCE1 and ANAPC10 wherein a decreased level of methylation in one or more of the genes relative to a reference level indicates that the patient has or is at risk of having an autism spectrum disorder.

In a further embodiment there is provided a method of detecting the severity of ASD in a patient (e.g., a patient previously diagnosed with ASD) comprising determining a methylation status in one or more genes in the patient sample selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, S100A13, DDX18, CD1D, ALKBH6, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p and POM121 wherein an increased or decreased level of methylation in one or more of the genes relative to a reference level indicates that the patient has or is at risk of having severe ASD. For example, a methylation status can be determined for one or more genes selected from the group consisting of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, POM121 (e.g., SPIN3, AIRE, ANKRD13C, GHSR, EIF3G, NOLC1, TPX2, BDNF, FAM149B1, ECD, OGG1, KATNAL1, ARMC1, LOC286002, PAIP2, MARCKS, DNAJC25, BLZF1, NME7, C12orf26, CCDC59, MCRS1, TTC22, FAM164A, PACRGL, CCDC58, TMEM126B, BLCAP, OTOF_{—}2, CA10, RASGRP4, MKLN1, RTDR1, STXBP4, COX11, C11orf83, SNCA, POLD4, LOC387647, ABCA5, NUBPL, SNRPF, CA10, PSCA, BC035336, POM121 wherein an increased level of methylation in one or more of the genes relative to a reference level indicates that the patient has or is at risk of having severe ASD. Likewise, in some aspects, a methylation status can be determined for one or more genes selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6 and S100A13 (e.g., NGDN, HAL, S100A1, OTOF_{—}1, HOXB6, LCK, ZNF248, THOC6, HCFC1R1, DDX18, and S100A13) wherein a decreased level of methylation in one or more of the genes relative to a reference level indicates that the patient has or is at risk of having severe ASD.

In some aspects, a method of the embodiments further comprises identifying a patient as having ASD, a biomarker of ASD or having a risk of developing ASD (or severe ASD). For example, a patient can be identified as having a biomarker of ASD if a sample from the patient is determined to have an increased level of DNA methylation relative to a reference at SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, POM121, or NLGN2; or a decreased level of DNA methylation relative to a reference at NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, or INGX. In certain aspects, identifying a patient as having a biomarker for ASD (or a biomarker of severe ASD) comprises reporting whether the patient has an ASD biomarker. For example, in some aspects, reporting comprises providing an electronic, written or oral report. In further aspects, a report is provided to the patient, a guardian of the patient, a health care worker (e.g., the patient's doctor), a hospital or an insurance company.

Certain aspects of the embodiments concern determining the methylation status in DNA. In some aspects, determining a methylation status comprises determining the proportion of DNA molecules in a sample that are methylated at a particular position or a set of positions in a region of DNA (e.g., a gene or a portion thereof). In some aspects, a level of methylation (e.g., the proportion of methylation in a gene region or proportion of DNA molecules comprising methylation at a DNA site or over a DNA region) is compared to a reference level. For example, the reference level can comprise a level of methylation from a patient know to have ASD, known to have mild ASD, known to have severe ASD or known to be free of ASD. In some aspects, the reference level comprises in a chart, database or instruction manual.

In certain aspects of the embodiments determining a methylation status comprises, performing methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, or bisulfate sequencing. Thus, in some aspects, determining methylation status comprises methylation specific PCR, real-time methylation specific PCR, quantitative methylation specific PCR (QMSP), or bisulfate sequencing. In certain aspects, a method according to the embodiments comprises treating DNA in or from a sample with bisulfate.

In still a further embodiment, there is provided a method for determining the effectiveness of a therapy in a patient being treated for ASD comprising determining a methylation status in one or more genes in a patient sample wherein an increased or decreased level of methylation in one or more of the genes relative to a reference level indicates that the therapy is effective. For example, the patient sample can be taken from a patient undergoing (or who has undergone) a therapy. The reference level can, for instance, be a level determined for a sample from a patient prior to therapy. In a further aspect, a method can comprise providing continued therapy, changing therapy, changing a therapy dosage or discontinuing therapy based the methylation status determined for the gene(s).

In some embodiments a method for treating a patient having an autism spectrum disorder (or at risk for having an autism spectrum disorder) is provided comprising administering a therapy to the patient, wherein the patient was previously determined to have an increased or decreased level of methylation in one or more of the genes selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121 relative to a reference level. For example, administering a therapy can comprise providing counseling or behavioral therapy or administering a pharmacological or electrostimulation therapy. Examples of pharmacological therapeutics for use according to embodiments include, without limitation, Milnacipran, Galantamine, Methylphenidate, Donepezil, STX209, Minocycline; Dimercaptosuccinic Acid (DMSA); Atomoxetine; Aripiprazole; Mecamylamine; Memantine and Acetyl-Choline Esterase Inhibitors. Electrostimulation therapies for use according to the embodiments include, without limitation, deep rTMS (repetitive transcranial magnetic stimulation) and transcranial direct current stimulation.

In a further embodiment there is provided a method for assessing the presence, severity, or an increased risk of, ASD in a patient comprising (a) obtaining a DNA sample from the patient; (b) identifying at least one genomic amplification interval, the interval comprising at least one CpG position within the recognition sequence of a methylation-sensitive endonuclease (MSE), where the CpG position is subject to differential methylation ASD; (c) amplifying the genomic interval in the presence and absence of the MSE; and (d) quantifying the amount of amplification product corresponding to the interval (with and without MSE treatment) to determine the proportion of DNA methylation in the genomic amplification interval, thereby assessing whether the patient has or is at risk of developing ASD (or sever ASD). For example, a method in accordance with the instant embodiment can comprise comparing the proportion of DNA methylation in the genomic amplification interval with a reference level of DNA methylation (e.g., for a known control or ASD sample) to determine the presence of or a risk for ASD. In certain aspects, a method further comprises identifying and amplifying 2, 3, 4, 5, 6, 7, 8, 9, 10 or more genomic amplification intervals. For example, one or more the genomic amplification intervals can comprise sequence from a gene or gene region listed in Table 1.

Methods in accordance with the embodiments can be applied to a variety of different types of patients. Preferably, the patient is a human patient. In certain aspects, the patient is less than about 20 years of age, such as less than 18, 16, 15, 14, 13, 12, 11, 10, 9, 8, 7, 6, 5, 4, 3, 2 or even less than 1 year of age. For example, the patient can be between about 1 month and about 12 months old, such about 2, 3, 4, 5, 6, 7, 8, 9 10 or 11 months of age. In some aspects, the subject may an in utero fetus.

Certain aspects of embodiments concern patient samples that comprise genomic DNA, such as blood, amniotic fluid, saliva, urine, fecal or a tissue sample. In certain aspects, a sample can be obtained directly from the patient (e.g., by drawing blood from the patient). In some further aspects, the sample can be a sample from a third party (e.g., a doctor) or can be from a tissue or blood bank.

In still a further embodiment, a kit is provided comprising a sealed container comprising primers or probes designed to detect methylation in one, two, three, four or more genes selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121. In further aspects, a kit can comprise a bisulfate reagent and/or one or more reagents for PCR, real-time PCR, or DNA sequencing.

In a further embodiment, a biochip is provided comprising an isolated nucleic acid comprising primers to detect methylation in at least 1, 2, 3, 4, 5, 6, 7, 8, 9, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or all 97 of the genes selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121. For example, biochip can comprise polynucleotide primers immobilized on a glass or silicon support.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

It is specifically contemplated that an individual component or element of a list may be specifically included or excluded from the claimed invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

### I. The Present Invention

Current methods for the diagnosis of ASD are highly subjective because they are based on behavioral assessments. Moreover, because the behaviors that are indicative of ASD develop as a child ages, it is difficult to diagnose the disorders or to determine their severity at an early age, when intervention could be most effective. The studies detailed in the instant application identify epigenetic changes present in the genome of patient with ASD. In particular, whole blood samples from a healthy patient and from patients with ASD were subjected to whole genome methylation analysis. These studies revealed alterations in the DNA methylation status in the promoter/enhancer regions of a number of genes that was specific to the patients with ASD. Importantly, two ASD patient samples were from identical twins, in which one of the pair was more severely afflicted with ASD than the other. When comparing the prevalence of methylation between the twins it was found that the relative levels of DNA hypermethylation (or hypomethylation) in the indicator genes was more profound in the twin with the most pronounced ASD symptoms. Thus, analysis of methylation status in the indicator genes can be used not only to detect ASD, but to assess ASD severity. Because the tests provided here do not require behavioral analysis they have the potential of detecting ASD at an early stage, when therapeutic intervention could reduce the severity of the disorder or even prevent emergence of ASD-associated behaviors.

### II. Detection of DNA Methylation

In one aspect, methods of the embodiments concern determining the presence of or an increased risk of developing ASD by determining the methylation status of genomic DNA of a subject. In certain aspects, methylation or hypermethylation in one, two, three, four, five, six, seven, or all of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 or POM121 indicates the presence of or an increased risk of ASD. Alternatively or additionally, lack of methylation or hypomethylation in one, two, three, four, five, six, seven, or all of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6 or S100A13 indicates the presence of or an increased risk of ASD.

In some aspects, methylation changes in the CES1, INGX and/or NLGN2 gene are used to determine a risk for autism spectrum disorder. For example, elevated methylation at positions in the CES1 or NLGN2 genes and/or decreased methylation at positions in the INGX can be indicative of an elevated risk of an ASD. Of particular interest is the identification of INGX hypomethylation in association with ASD given that genetic or epigenetic changes on the X chromosome could explain why ASD is more prevalent in male children (who have only one X chromosome copy) as opposed to females. Thus, INGX hypomethylation could contribute the observed disparity in male/female prevalence of ASD.

Methylation typically occurs in a CpG containing nucleic acid. The CpG containing nucleic acid may be present in, e.g., in a CpG island, a CpG doublet, a promoter, an intron, or an exon. For instance, in the genetic regions provided herein the potential methylation sites encompass the promoter/enhancer regions of the indicated genes. Thus, the regions can begin upstream of a gene promoter and extend downstream into the transcribed region. In certain specific aspects DNA methylation is determined one or more of the regions of the genes. For example, for the regions provided in Table 1 methylation can be determined at each CpG and an average calculated for each region.

TABLE 1
Exemplary genomic regions for analysis

| | | Chromosome | Chromosome |
|---|---|---|---|
| gene ID | chromosome | start of region* | end of region* |
| NGDN | chr14 | 23007737 | 23009737 |
| HAL | chr12 | 94913202 | 94915202 |
| S100A1 | chr1 | 151866339 | 151868339 |
| OTOF “OTOF_1”^{#} | chr2 | 26634070 | 26636070 |
| MTBP | chr8 | 121525828 | 121527828 |
| MRPL13 | chr8 | 121525846 | 121527846 |
| HOXB6 | chr17 | 44036333 | 44038333 |
| KAZN | chr1 | 15127882 | 15129882 |
| MORC2-AS1 | chr22 | 29647294 | 29649294 |
| LCK | chr1 | 32488426 | 32490426 |
| ZNF248 | chr10 | 38185492 | 38187492 |
| ANAPC7 | chr12 | 109324918 | 109326918 |
| SEC1 | chr19 | 53840263 | 53842263 |
| THOC6 | chr16 | 3013032 | 3015032 |
| HCFC1R1 | chr16 | 3013288 | 3015288 |
| ABCE1 | chr4 | 146237605 | 146239605 |
| ANAPC10 | chr4 | 146237818 | 146239818 |
| DDX18 | chr2 | 118287724 | 118289724 |
| CD1D | chr1 | 156415360 | 156417360 |
| ALKBH6 | chr19 | 41195981 | 41197981 |
| S100A13 | chr1 | 151865741 | 151867741 |
| SPIN3 | chrX | 57037713 | 57039713 |
| LALBA | chr12 | 47249096 | 47251096 |
| EIF4A1 | chr17 | 7417754 | 7419754 |
| CENPF | chr1 | 212842154 | 212844154 |
| AIRE | chr21 | 44529190 | 44531190 |
| ANKRD13C | chr1 | 70592005 | 70594005 |
| GHSR | chr3 | 173647897 | 173649897 |
| EIF3G | chr19 | 10090599 | 10092599 |
| NOLC1 | chr10 | 103900922 | 103902922 |
| TPX2 | chr20 | 29789564 | 29791564 |
| BDNF | chr11 | 27678756 | 27680756 |
| FAM149B1 | chr10 | 74596859 | 74598859 |
| ECD | chr10 | 74596882 | 74598882 |
| OGG1 | chr3 | 9765627 | 9767627 |
| KATNAL1 | chr13 | 29778584 | 29780584 |
| IGFBP5 | chr2 | 217267517 | 217269517 |
| MRPS15 | chr1 | 36701627 | 36703627 |
| HIST1H4K | chr6 | 27906284 | 27908284 |
| CMYA5 | chr5 | 79020414 | 79022414 |
| ARMC1 | chr8 | 66707986 | 66709986 |
| LOC286002 | chr7 | 107087315 | 107089315 |
| PAIP2 | chr5 | 138705032 | 138707032 |
| MARCKS | chr6 | 114284219 | 114286219 |
| DNAJC25-GNG10 | chr9 | 113432486 | 113434486 |
| BLZF1 | chr1 | 167602810 | 167604810 |
| NME7 | chr1 | 167602817 | 167604817 |
| C12orf26 | chr12 | 81275330 | 81277330 |
| CCDC59 | chr12 | 81275454 | 81277454 |
| MCRS1 | chr12 | 48247178 | 48249178 |
| RBM4B | chr11 | 66200851 | 66202851 |
| MIR548H4 | chr16 | 79131354 | 79133354 |
| TEKT1 | chr17 | 6674784 | 6676784 |
| TMEM184C | chr4 | 148756988 | 148758988 |
| TTC22 | chr1 | 55038529 | 55040529 |
| FAM164A | chr8 | 79739836 | 79741836 |
| PACRGL | chr4 | 20310133 | 20312133 |
| CCDC58 | chr3 | 123583764 | 123585764 |
| TMEM126B | chr11 | 85016309 | 85018309 |
| BLCAP | chr20 | 35582020 | 35584020 |
| LOC100131564 | chr1 | 93583065 | 93585065 |
| GTF3C2 | chr2 | 27432372 | 27434372 |
| RBP5 | chr12 | 7171733 | 7173733 |
| NVL | chr1 | 222583495 | 222585495 |
| NARG2 | chr15 | 58557636 | 58559636 |
| PTPLAD2 | chr9 | 21020635 | 21022635 |
| OTOF “OTOF_2”^{#} | chr2 | 26553414 | 26555414 |
| CA10 | chr17 | 47591160 | 47593160 |
| RASGRP4 | chr19 | 43607785 | 43609785 |
| CES1 | chr16 | 54423576 | 54425576 |
| ALX1 | chr12 | 84197166 | 84199166 |
| C11orf48 | chr11 | 62194701 | 62196701 |
| LYRM7 | chr5 | 130533539 | 130535539 |
| MGAT5B | chr17 | 72379323 | 72381323 |
| MKLN1 | chr7 | 130444394 | 130446394 |
| RTDR1 | chr22 | 21813241 | 21815241 |
| STXBP4 | chr17 | 50400053 | 50402053 |
| COX11 | chr17 | 50400124 | 50402124 |
| C11orf83 | chr11 | 62194817 | 62196817 |
| SNCA | chr4 | 90977470 | 90979470 |
| POLD4 | chr11 | 66876593 | 66878593 |
| CLNS1A | chr11 | 77025499 | 77027499 |
| DNAJC19 | chr3 | 182189224 | 182191224 |
| JAM2 | chr21 | 25932459 | 25934459 |
| ITGA4 | chr2 | 182028863 | 182030863 |
| DGUOK | chr2 | 74006460 | 74008460 |
| LOC387647 | chr10 | 29737506 | 29739506 |
| ABCA5 | chr17 | 64833918 | 64835918 |
| NUBPL | chr14 | 31099341 | 31101341 |
| SNRPF | chr12 | 94775839 | 94777839 |
| CA10 | chr17 | 47591376 | 47593376 |
| PSCA | chr8 | 143757876 | 143759876 |
| BC035336 | chr18 | 72368599 | 72370599 |
| DDX11 | chr12 | 9491035 | 9493035 |
| KCNIP4 | chr5 | 169712458 | 169714458 |
| PDIA3p | chr1 | 145115053 | 145117053 |
| POM121 | chr7 | 71986871 | 71988871 |
| INGX | chrX | 70628329 | 70630329 |
| NLGN2 | chr17 | 7251225 | 7253225 |

^{#}OTOF_1 and OTOF_2 refer to two different regions of the OTOF gene. The boundries of these regions are provided in the chromosome start and end positions listed in Table 1.
*The chromosomal start and end positions are based on the number in the March 2006 human reference sequence (NCBI Build 36.1) produced by the International Human Genome Sequencing Consortium. See NCBI accession nos. NC_000001.9 (Chrom. 1); NC_000002.10 (Chrom. 2); NC_000003.10 (Chrom. 3); NC_000004.10 (Chrom. 4); NC_000005.8 (Chrom. 5); NC_000006.10 (Chrom. 6); NC_000007.12 (Chrom. 7); NC_000008.9 (Chrom.
8); NC_000009.10 (Chrom. 9); NC_000010.9 (Chrom. 10); NC_000011.8 (Chrom. 11); NC_000012.10 (Chrom. 12); NC_000013.9 (Chrom. 13); NC_000014.7 (Chrom. 14); NC_000015.8 (Chrom. 15); NC_000016.8 (Chrom. 16); NC_000017.9 (Chrom. 17); NC_000018.8 (Chrom. 18); NC_000019.8 (Chrom. 19); NC_000020.9 (Chrom. 20); NC_000021.7 (Chrom. 21); NC_000022.9 (Chrom. 22); NC_000023.9 (Chrom. X); and NC_000024.8 (Chrom. Y), each of which is incorporated herein by reference in its entirety.

Other methods which may be used to detect methylation of a gene include, e.g., Combined Bisulfite Restriction Analysis (COBRA) and methylated DNA immunoprecipitation (MeDIP) (Xiong et al., 1997; Weber et al., 2005; Keshet et al., 2006). In some embodiments direct sequencing may be used to detect methylation (Frommer et al., 1992).

A variety of methods for detecting the presence, absence, or amount of methylation in a gene are known in the art and may be used to evaluate the methylation status of one or more genes as described herein. For example, methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, or bisulfite sequencing may be used to detect and/or quantify methylation in a gene.

Many methods for detecting the presence, absence, or amount of methylation involve exposing genomic DNA to bisulfite. Exposure of DNA, such as genomic DNA, to bisulfite does not effect methylated cytosine in a CpG, while unmethylated cytosines are changed to uracil. Thus, many methods for detecting methylation of a gene involve the treatment of DNA with bisulfite, and subsequent analysis of the resulting nucleotide. Techniques for bisulfate conversion of DNA are well known in the art and commercial kits that provide optimized conversion efficiency are available (e.g., the EZ DNA Methylation-Startup™ kit available from Zymo Research Corp., Irvine, Calif.). Various methods for detecting methylation are disclosed, e.g., in PCT Pub. No. WO/2010/114821; PCT Pub. No. WO/2011/109529; U.S. 2011/0046009, U.S. 2010/0304992, U.S. Pat. No. 5,786,146; Fraga, 2002; El-Maarri, 2003; Laird, 2003; and Callinan, 2006, which are incorporated herein by reference in their entirety.

A. Methylation Specific PCR (MSP)

Methylation specific PCR typically utilizes bisulfite treatment of a nucleic acid to detect methylation. For a base sequence modified by bisulfite treatment, PCR primers corresponding to regions in which a 5′-CpG-3′ base sequence is present may be constructed. For example, two kinds of primers corresponding to the methylated case and the unmethylated case may be generated. More specifically, primer pairs may thus be designed to be “methylated-specific” by including sequences complementing only unconverted 5-methylcytosines, or, on the converse, “unmethylated-specific”, complementing thymines converted from unmethylated cytosines. Methylation is determined by the ability of the specific primer to achieve amplification. When genomic DNA is modified with bisulfite and then subjected to PCR using the two kinds of primers, if DNA is methylated, then a PCR product can be made from the DNA from a primer corresponding to the methylated base sequence. In contrast, if that region of the gene is unmethylated, a PCR product can be made from the DNA based on a primer corresponding to the unmethylated base sequence. The methylation of DNA can be qualitatively analyzed, e.g., using agarose gel electrophoresis.

In some embodiments, placing the CpG pair at the 3′-end of a primer may improve the sensitivity. The initial report using MSP described sufficient sensitivity to detect methylation of 0.1% of alleles.

B. Real-Time Methylation-Specific PCR

Real-time methylation-specific PCR generally involves a real-time measurement method, such as real-time PCR, modified from methylation-specific PCR. The method may involve treating genomic DNA with bisulfite, and utilizing methylated-specific and unmethylated-specific PCR primers in combination with real-time PCR. The method may involve performing detection using a TaqMan® probe complementary to the amplified base sequence, or detection using Sybergreen®. Generally, real-time methylation-specific PCR can quantitatively analyze DNA. A standard curve may be prepared using an in vitro methylated DNA sample, and for standardization, a gene having no 5′-CpG-3′ sequence in the base sequence may be amplified as a negative control; in this way the degree of methylation of a gene may be calculated.

The MethyLight method is an example of a method that is based on MSP, but can provide a quantitative analysis using real-time PCR (Eads et al., 2000). Methylated-specific primers are typically used, and a methylated-specific fluorescence reporter probe may also be used to anneal to the amplified region. Alternately, the primers or probe can be designed without methylation specificity, e.g., if discrimination is desired between the CpG pairs within the involved sequences. Quantification can be calculated in comparison to a methylated reference DNA. This protocol may be modified to increase the specificity of the PCR for successfully bisulfite-converted DNA by using an additional probe to bisulfite-unconverted DNA to quantify a non-specific amplification (Rand et al., 2002).

Optimized single-step methylation specific RT-PCR can also be achieved using a OneStep qMethyl™ kit (available from Zymo Research Corp, Irvine, Calif.). This system can be used for the detection of locus-specific DNA methylation by selective amplification of a methylated regions of DNA. This is accomplished by splitting any DNA to be tested into two parts: a “Test Reaction” and a “Reference Reaction”. DNA in the Test Reaction is digested with Methylation Sensitive Restriction Enzymes (MSREs) while DNA in the Reference Reaction is not digested. The DNA from both samples is then amplified using real-time PCR in the presence of a fluorescent dye, such as SYTO®9, and then quantified. Because of the simplicity of the method such as system can be used to examine multiple sites of potential methylation (e.g., 10, 50, 100, 1,000 or more sites) essentially simultaneously and can provide a quantitative readout of methylation at each site.

Melting-curve analysis (Mc-MSP) may also be used to quantify the amount of methylation in a DNA, and generally involves the evaluation of MSP-amplified DNA (Akey et al., 2002). This method generally involves amplifying bisulfate-converted DNA with both methylated-specific and unmethylated-specific primers, and determining the quantitative ratio of the two products by comparing the differential peaks generated in a melting-curve analysis. Some Mc-MSP methods may use both real-time quantification and melting analysis, which may be particularly useful, e.g., for sensitive detection of low-level methylation (Kristensen et al., 2008).

C. DNA Sequencing

DNA sequencing, including single molecule sequencing, such as pyrosequencing or sequencing by ligation (e.g., SOLiD™), may be used to detect the presence, absence, or amount of methylation of a gene. Such sequencing may be used to analyze bisulfite-treated DNA without the need for methylation-specific PCR (Colella et al., 2003; Tost et al., 2003). Sequencing is then employed (with or without first amplifying the sequence by PCR) to determine the bisulfate-converted sequence of specific CpG sites in the region. The ratio of C-to-T at individual sites can be determined quantitatively based on the amount of C and T incorporation during the sequence extension. Pyrosequencing, for example, may be particularly effective for high-throughput screening methods or for examining large regions of genomic DNA. In some embodiments, allele-specific primers may be used that incorporate single-nucleotide polymorphisms into the sequence of the sequencing primer (Wong et al., 2006).

D. Base-Specific Cleavage/MALDI-TOF

Base-specific cleavage/MALDI-TOF may be used to detect methylation of a gene (Ehrich et al. 2005). This method typically involves using in vitro transcription of the region of interest into RNA (e.g., by adding an RNA polymerase promoter site to the PCR primer in the initial amplification), and then cleavage of the RNA transcript at base-specific sites with RNase A. Since RNase A can cleave RNA specifically at cytosine and uracil ribonucleotides, base-specificity is achieved by adding incorporating cleavage-resistant dTTP when cytosine-specific (C-specific) cleavage is desired, and incorporating dCTP when uracil-specific (U-specific) cleavage is desired. The cleaved fragments can then be analyzed by MALDI-TOF. Bisulfite treatment can result in either introduction/removal of cleavage sites by C-to-U conversions or shift in fragment mass by G-to-A conversions in the amplified reverse strand. C-specific cleavage can cut specifically at the methylated CpG sites. By analyzing the sizes of the resulting fragments, it is possible to determine the specific pattern of DNA methylation of CpG sites within the region, rather than determining the extent of methylation of the region as a whole.

E. Methylation-Sensitive Single-Strand Conformation Analysis (MS-SSCA)

Methylation-sensitive single-strand conformation analysis (MS-SSCA) may be used to detect methylation in a gene. This method is based on the single-strand conformation polymorphism analysis (SSCA) method, which has been used for single-nucleotide polymorphism (SNP) analysis (Bianco et al., 1999). SSCA can differentiate between single-stranded DNA fragments of identical size but distinct sequence based on differential migration in non-denaturating electrophoresis. In MS-SSCA, this approach can be used to distinguish between bisulfite-treated, PCR-amplified regions containing the CpG sites of interest. Bisulfite treatment of DNA can make C-to-T conversions in most regions, which can result in high sensitivity. MS-SSCA can provide semi-quantitative analysis of the degree of DNA methylation based on the ratio of band intensities. This method may be used to evaluate most or all CpG sites in a DNA region of interest.

F. High Resolution Melting Analysis (HRM)

High-resolution melting analysis (HRM) is a real-time PCR-based technique which may be used to detect methylation, e.g., by differentiating converted from unconverted bisulfite-treated DNA (Wojdacz and Dobrovic, 2007). PCR amplicons can be analyzed directly by temperature ramping and resulting liberation of an intercalating fluorescent dye during melting. The degree of methylation, as represented by the C-to-T content in the amplicon, can be used to determine the rapidity of melting and consequent release of the dye. This method can allow for detecting methylation in a gene in a single-tube assay.

G. Methylation-Sensitive Single-Nucleotide Primer Extension (MS-SnuPE)

Methylation-sensitive single-nucleotide primer extension (MS-SnuPE) may be used to detect methylation of a gene (Gonzalgo and Jones, 1997). DNA is bisulfite-converted, and bisulfate-specific primers are annealed to the sequence up to the base pair immediately before the CpG of interest. The primer is allowed to extend one base pair into the C (or T) using DNA polymerase terminating dideoxynucleotides, and the ratio of C to T is determined quantitatively. The C:T ratio may be determined by a variety of techniques including, e.g., radioactive ddNTPs incorporation, fluorescence-based methods, pyrosequencing, matrix-assisted laser desorption ionization/time-of-flight (MALDI-TOF) mass spectrometry, or ion pair reverse-phase high-performance liquid chromatography (IP-RP-HPLC) has also been used to distinguish primer extension products (Uhlmann et al., 2002; Matin et al., 2002).

H. Detection of Differential Methylation-Methylation Sensitive Restriction Endonuclease

Detection of methylation in a gene can be accomplished, in some embodiments, by contacting a nucleic acid sample with a methylation sensitive restriction endonuclease that cleaves only unmethylated CpG sites under conditions and for a time to allow cleavage of unmethylated nucleic acid. In a separate reaction, the sample may be further contacted with an isoschizomer of the methylation sensitive restriction endonuclease that cleaves both methylated and unmethylated CpG-sites under conditions and for a time to allow cleavage of methylated nucleic acid. Specific primers may be added to the nucleic acid sample under conditions and for a time to allow nucleic acid amplification to occur. The presence of amplified product in the sample digested with methylation sensitive restriction endonuclease but absence of an amplified product in sample digested with an isoschizomer of the methylation sensitive restriction enzyme endonuclease that cleaves both methylated and unmethylated CpG-sites can indicate that methylation has occurred at the nucleic acid region being assayed. Lack of amplified product in the sample digested with methylation sensitive restriction endonuclease together with lack of an amplified product in the sample digested with an isoschizomer of the methylation sensitive restriction enzyme endonuclease that cleaves both methylated and unmethylated CpG-sites can indicate that methylation has not occurred at the nucleic acid region being assayed.

I. Microarray and DNA Chip-Based Methods

Microarray-based or DNA Chip-based methods may be used to detect methylation, e.g., in bisulfate-treated DNA (Adorján et al., 2002). An oligonucleotide microarray or DNA chip may be produced using oligonucleotide pairs targeting CpG sites of interest, e.g., with one or more primer complementary to a methylated sequence, and another primer complimentary to a C-to-U-converted unmethylated sequence. The oligonucleotides may be bisulfate-specific to prevent binding to any DNA which has been incompletely converted by bisulfate. Microarray-based methods include, e.g., the Illumina Methylation Assay. In some embodiments, a microarray or DNA chip may be configured to detect methylation in one, two, three, four, five, six, seven, or all of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121.

J. Indirect Detection

While the methodologies detailed above involve direct assessment of DNA methylation status, it will be recognized that methylation can also be indirectly assessed. For example, indirect assessment can comprise correlating methylation and gene expression levels, thus methylation status can be determined by determining the expression level of the indicated gene. Accordingly, in one embodiment a method if provided for detecting the presence of, or an increased risk of, an autism spectrum disorder in a patient by determining the expression level of one or more genes selected from the group consisting of NGDN, HAL, S100A1, OTOF, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121. Any of a variety of methods well known in the art can be used for determining an expression level of gene including determining the RNA expression level (e.g., by quantitative hybridization or reverse transcription PCR) or the protein expression level (e.g., by immunoblot or ELISA)

### III. Kits

The technology herein includes kits for evaluating presence, absence, or amount of methylation in a NGDN, HAL, S100A1, OTOF, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121 gene in a sample (e.g., in the promoter region of such a gene). A “kit” refers to a combination of physical elements. For example, a kit may include, for example, one or more components such as probes, including without limitation specific primers, enzymes, reaction buffers, an instruction sheet, and other elements useful to practice the technology described herein. The kits may include one or more primers, such as primers for PCR, to detect methylation of one or more of the genes as described herein. These physical elements can be arranged in any way suitable for carrying out the invention.

Kits for analyzing methylation of one or more genes may include, for example, a set of oligonucleotide probes for detecting methylation in NGDN, HAL, S100A1, OTOF, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121. The probes can be provided on a solid support, as in an array (e.g., a microarray), or in separate containers. The kits can include a set of oligonucleotide primers useful for amplifying a set of genes described herein, such as to perform PCR analysis. Kits can include further buffers, enzymes, labeling compounds, and the like. Any of the compositions described herein may be comprised in a kit. The kit may further include water and hybridization buffer to facilitate hybridization of the two nucleic acid strands.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the invention. Such kits, however, are not limited to the particular items identified above and may include any reagent used for the manipulation or characterization of the methylation of a gene.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### IV. Treatment

Certain aspects of the embodiments concerns methods for treating a subject having or at risk of developing ASD, such as subject comprising the alterations in DNA methylation described herein. In certain aspects, the subject for treatment comprise alterations in DNA methylation in accordance with the embodiments, but does not yet exhibit the behavioral abnormalities diagnostic of autism. Treatment of such subjects can include any of the behavioral therapies and/or pharmacological therapies used for subject having ASD. For example, pharmacotherapies of disruptive and aggressive behaviors such as antipsychotic medications or selective serotonin reuptake inhibitors (SSRIs) for anxiety, depression and repetitive behaviors (see, e.g., Cook et al., 1996; Hollander et al., 2005). For example, SSRIs that may be used according to the embodiments include, without limitation, citalopram, dapoxetine, escitalopram, fluoxetine, fluvoxamine, paroxetine, sertraline and/or vilazodone.

### V. Definitions

As used herein, a “methylation sensitive restriction endonuclease” is a restriction endonuclease that includes CG as part of its recognition site and has altered activity when the C is methylated as compared to when the C is not methylated (e.g., Sma I). Non-limiting examples of methylation sensitive restriction endonucleases include MspI, HpaII, BssHII, BstUI, SacII and EagI and Nod. An “isoschizomer” of a methylation sensitive restriction endonuclease is a restriction endonuclease that recognizes the same recognition site as a methylation sensitive restriction endonuclease but cleaves both methylated CGs and unmethylated CGs, such as for example, MspI.

As used herein the “hypermethylation” indicates an increase in the presence of methylation in a sample relative to a reference level. For example, hypermethylation can refer to an increased number of methylated positions in a region of DNA or an increased proportion of DNA molecules in a sample that comprise methylation at a particular position or in a region of DNA sequence.

As used herein the “hypomethylation” indicates decrease in the presence of methylation in a sample relative to a reference level. For example, hypomethylation can refer to a decreased number of methylated positions in a region of DNA or decreased proportion of DNA molecules in a sample that comprise methylation at a particular position or in a region of DNA sequence.

As used herein the term “gene” refers to a region of genomic DNA encoding and controlling expression of a particular RNA or polypeptide (such as sequences coding for exons, intervening introns and associated expression control sequences) and its flanking sequence. Thus, in some aspects, a gene is defined by the regions encoding the genes listed in Table 1. It is, however, recognized in the art that methylation in a particular region (e.g., at a given CpG position or in an amplification interval) is generally indicative of the methylation status at proximal genomic sites. Accordingly, determining a methylation status a particular gene can comprise determining a methylation status at a site or sites within about 100, 50, or 25 KB of a named gene.

As used herein the term “genomic amplification interval” refers to a region of genomic DNA that can be amplified by PCR. As used herein an amplification interval comprises at least one CpG position that is a potential site of methylation. In some cases, the amplification interval comprises 2, 3, 4 or more potential sites of CpG methylation (e.g., wherein the CpG is in a sequence recognized by an MSE). In general an amplification interval is less than about 1,200 bp, such as between about 50 bp and 100, 200, 300, 400 or 500 bp.

As used herein “determining a methylation status” for an indicated gene means determining whether one of more position in the DNA of the gene is methylated. Thus, in certain aspects, determining a methylation status for a gene comprises determining the methylation status at least 1, 2, 3, 4, 5, 6, 7, 8, 9, 10, 15, 20, 25, 30, 35, 40, 45, 50 or more sites of potential DNA methylation.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Whole Genome DNA Methylation Analysis in ASD and Control Subjects

Genomic DNA was extracted from the three blood samples of ASD (samples “A” and “J”) and control subjects (sample “C”). Notably of the two ASD patients sample A was from a patient with more severe symptoms than sample J. 300 ng of gDNA was processed to prepare the correspondent RRBS (reduced representative bisulfate sequencing) library which then subjected to next generation sequencing using illumine HiSeq2000 genome analyzer. Sequence reads were first aligned to the reference genome and further analysis were performed by a bioinformatic pipeline that scores epigenetic alterations according to strength and significance and links them to potentially affected genes. A comprehensive set of regions of interest, such as gene promoters, CpG islands, exon, intron and enhancer were collected. For each of these regions, the number of methylated and unmethylated CpG observations was determined, and a P value was assigned using Fisher's exact test. Once all P values were calculated, multiple testing correction was performed separately for each region type using the q-value method, which controls the false discovery rate to be below a user-specified threshold (typically 10%).

TABLE 2
Associate between ASD methylation status in the promoter/enhancer regions of genes.

| | | | | | | | mDifference | |
|---|---|---|---|---|---|---|---|---|
| gene ID | chrom | chromstart | chromend | C-mRatio | A-mRatio | J-mRatio | C_VS_A | pValue_C_vs_A |
| NGDN | chr14 | 23007737 | 23009737 | 0.3336397 | 0 | 0.076923 | 3.3364e−01 | 5.7023e−04 |
| HAL | chr12 | 94913202 | 94915202 | 0.8868687 | 0.537037 | 0.760417 | 3.4983e−01 | 2.3999e−04 |
| S100A1 | chr1 | 151866339 | 151868339 | 0.5666667 | 0.2117347 | 0.240278 | 3.5493e−01 | 2.0537e−02 |
| OTOF | chr2 | 26634070 | 26636070 | 0.7619048 | 0.3818342 | 0.670878 | 3.8007e−01 | 2.1209e−02 |
| (“OTOF_1”) | | | | | | | | |
| MTBP | chr8 | 121525828 | 121527828 | 0.3966667 | 0.012987 | 0.021667 | 3.8368e−01 | 2.8265e−02 |
| MRPL13 | chr8 | 121525846 | 121527846 | 0.3966667 | 0.012987 | 0.021667 | 3.8368e−01 | 2.8265e−02 |
| HOXB6 | chr17 | 44036333 | 44038333 | 0.8333333 | 0.4444444 | 0.281818 | 3.8889e−01 | 2.3886e−04 |
| KAZN | chr1 | 15127882 | 15129882 | 0.9818182 | 0.5833333 | 0.833333 | 3.9848e−01 | 8.9144e−03 |
| MORC2- | chr22 | 29647294 | 29649294 | 0.4 | 0 | 0.305238 | 4.0000e−01 | 1.5480e−03 |
| AS1 | | | | | | | | |
| LCK | chr1 | 32488426 | 32490426 | 0.5397808 | 0.1333333 | 0.575 | 4.0645e−01 | 3.2783e−03 |
| ZNF248 | chr10 | 38185492 | 38187492 | 0.4363636 | 0.0246914 | 0.047786 | 4.1167e−01 | 2.4143e−02 |
| ANAPC7 | chr12 | 109324918 | 109326918 | 0.4482601 | 0.0164835 | 0.064713 | 4.3178e−01 | 6.7307e−07 |
| SEC1 | chr19 | 53840263 | 53842263 | 0.49 | 0.0535714 | 0.048718 | 4.3643e−01 | 2.6475e−02 |
| THOC6 | chr16 | 3013032 | 3015032 | 0.7142857 | 0.2777778 | 0.25 | 4.3651e−01 | 3.0664e−02 |
| HCFC1R1 | chr16 | 3013288 | 3015288 | 0.7142857 | 0.2777778 | 0.25 | 4.3651e−01 | 3.0664e−02 |
| ABCE1 | chr4 | 146237605 | 146239605 | 0.462585 | 0.0244898 | 0.074405 | 4.3810e−01 | 3.8602e−03 |
| ANAPC10 | chr4 | 146237818 | 146239818 | 0.462585 | 0.0244898 | 0.074405 | 4.3810e−01 | 3.8602e−03 |
| DDX18 | chr2 | 118287724 | 118289724 | 0.4779412 | 0.0073529 | 0.008553 | 4.7059e−01 | 2.5541e−02 |
| CD1D | chr1 | 156415360 | 156417360 | 0.5 | 0 | 0.4375 | 5.0000e−01 | 1.5128e−03 |
| ALKBH6 | chr19 | 41195981 | 41197981 | 0.625 | 0.1060606 | 0.020833 | 5.1894e−01 | 3.1310e−02 |
| S100A13 | chr1 | 151865741 | 151867741 | 0.752381 | 0.1435185 | 0.257407 | 6.0886e−01 | 1.2472e−03 |
| SPIN3 | chrX | 57037713 | 57039713 | 0 | 1 | 0 | −1.0000e+00 | 3.0650e−07 |
| LALBA | chr12 | 47249096 | 47251096 | 0 | 1 | 0.85 | −1.0000e+00 | 4.7619e−02 |
| EIF4A1 | chr17 | 7417754 | 7419754 | 0.05641 | 0.391177 | 0.1381 | −3.3477e−01 | 1.0658e−02 |
| CENPF | chr1 | 212842154 | 212844154 | 0.011765 | 0.347222 | 0.00794 | −3.3546e−01 | 2.9411e−02 |
| AIRE | chr21 | 44529190 | 44531190 | 0.366172 | 0.702778 | 0.29068 | −3.3661e−01 | 4.0151e−07 |
| ANKRD13C | chr1 | 70592005 | 70594005 | 0.072381 | 0.410973 | 0.06466 | −3.3859e−01 | 2.7708e−03 |
| GHSR | chr3 | 173647897 | 173649897 | 0.090062 | 0.428818 | 0.11293 | −3.3876e−01 | 3.9291e−03 |
| EIF3G | chr19 | 10090599 | 10092599 | 0 | 0.339731 | 0.02143 | −3.3973e−01 | 3.5988e−02 |
| NOLC1 | chr10 | 103900922 | 103902922 | 0 | 0.340553 | 0.01068 | −3.4055e−01 | 2.4827e−03 |
| TPX2 | chr20 | 29789564 | 29791564 | 0.048214 | 0.389569 | 0.20985 | −3.4135e−01 | 4.0095e−02 |
| BDNF | chr11 | 27678756 | 27680756 | 0 | 0.341991 | 0.16583 | −3.4199e−01 | 1.3341e−02 |
| FAM149B1 | chr10 | 74596859 | 74598859 | 0.009524 | 0.351563 | 0.00694 | −3.4204e−01 | 9.5133e−04 |
| ECD | chr10 | 74596882 | 74598882 | 0.009524 | 0.351563 | 0.00694 | −3.4204e−01 | 9.5133e−04 |
| OGG1 | chr3 | 9765627 | 9767627 | 0.090909 | 0.433607 | 0.13786 | −3.4270e−01 | 4.0264e−03 |
| KATNAL1 | chr13 | 29778584 | 29780584 | 0 | 0.348558 | 0.14744 | −3.4856e−01 | 5.3831e−03 |
| IGFBP5 | chr2 | 217267517 | 217269517 | 0.030983 | 0.382353 | 0.22569 | −3.5137e−01 | 2.3108e−03 |
| MRPS15 | chr1 | 36701627 | 36703627 | 0.030794 | 0.384868 | 0.00615 | −3.5407e−01 | 1.7727e−04 |
| HIST1H4K | chr6 | 27906284 | 27908284 | 0.053333 | 0.408333 | 0.38636 | −3.5500e−01 | 5.6254e−03 |
| CMYA5 | chr5 | 79020414 | 79022414 | 0.377789 | 0.733631 | 0.65648 | −3.5584e−01 | 8.8608e−09 |
| ARMC1 | chr8 | 66707986 | 66709986 | 0.027273 | 0.385417 | 0.02479 | −3.5814e−01 | 4.0644e−02 |
| LOC286002 | chr7 | 107087315 | 107089315 | 0.010417 | 0.373099 | 0.0498 | −3.6268e−01 | 5.1478e−03 |
| PAIP2 | chr5 | 138705032 | 138707032 | 0 | 0.368421 | 0 | −3.6842e−01 | 4.9685e−06 |
| MARCKS | chr6 | 114284219 | 114286219 | 0.020032 | 0.4 | 0.19815 | −3.7997e−01 | 1.9381e−03 |
| DNAJC25- | chr9 | 113432486 | 113434486 | 0.059354 | 0.442027 | 0.07714 | −3.8267e−01 | 1.3072e−04 |
| GNG10 | | | | | | | | |
| BLZF1 | chr1 | 167602810 | 167604810 | 0 | 0.385417 | 0.14286 | −3.8542e−01 | 4.3811e−02 |
| NME7 | chr1 | 167602817 | 167604817 | 0 | 0.385417 | 0.14286 | −3.8542e−01 | 4.3811e−02 |
| C12orf26 | chr12 | 81275330 | 81277330 | 0.041939 | 0.427444 | 0.15975 | −3.8550e−01 | 2.8225e−06 |
| CCDC59 | chr12 | 81275454 | 81277454 | 0.041939 | 0.427444 | 0.15975 | −3.8550e−01 | 2.8225e−06 |
| MCRS1 | chr12 | 48247178 | 48249178 | 0.025483 | 0.411261 | 0.04464 | −3.8578e−01 | 8.5914e−03 |
| RBM4B | chr11 | 66200851 | 66202851 | 0.019676 | 0.40625 | 0.21875 | −3.8657e−01 | 2.4576e−04 |
| MIR548H4 | chr16 | 79131354 | 79133354 | 0.030135 | 0.42037 | 0.3 | −3.9024e−01 | 2.6783e−03 |
| TEKT1 | chr17 | 6674784 | 6676784 | 0 | 0.393939 | 0.02208 | −3.9394e−01 | 4.2749e−03 |
| TMEM184C | chr4 | 148756988 | 148758988 | 0.017647 | 0.412843 | 0.08386 | −3.9520e−01 | 3.9724e−02 |
| TTC22 | chr1 | 55038529 | 55040529 | 0.206101 | 0.609477 | 0.12013 | −4.0338e−01 | 2.0953e−02 |
| FAM164A | chr8 | 79739836 | 79741836 | 0.068027 | 0.471429 | 0.05014 | −4.0340e−01 | 3.2982e−04 |
| PACRGL | chr4 | 20310133 | 20312133 | 0.007576 | 0.411111 | 0.04545 | −4.0354e−01 | 8.7875e−03 |
| CCDC58 | chr3 | 123583764 | 123585764 | 0.091346 | 0.501166 | 0.17949 | −4.0982e−01 | 2.1516e−04 |
| TMEM126B | chr11 | 85016309 | 85018309 | 0.019536 | 0.430622 | 0.08862 | −4.1109e−01 | 6.6790e−03 |
| BLCAP | chr20 | 35582020 | 35584020 | 0.353846 | 0.766667 | 0.45503 | −4.1282e−01 | 2.7925e−05 |
| LOC100131564 | chr1 | 93583065 | 93585065 | 0.020313 | 0.434667 | 0.02745 | −4.1435e−01 | 3.9234e−03 |
| GTF3C2 | chr2 | 27432372 | 27434372 | 0.083333 | 0.501389 | 0.12658 | −4.1806e−01 | 2.7179e−02 |
| RBP5 | chr12 | 7171733 | 7173733 | 0.166667 | 0.589286 | 0.375 | −4.2262e−01 | 1.1619e−02 |
| NVL | chr1 | 222583495 | 222585495 | 0.017857 | 0.444444 | 0 | −4.2659e−01 | 8.1534e−03 |
| NARG2 | chr15 | 58557636 | 58559636 | 0 | 0.428571 | 0.01786 | −4.2857e−01 | 6.5063e−03 |
| PTPLAD2 | chr9 | 21020635 | 21022635 | 0.3125 | 0.742857 | 0.26174 | −4.3036e−01 | 3.2471e−07 |
| OTOF | chr2 | 26553414 | 26555414 | 0.1 | 0.533333 | 0.52408 | −4.3333e−01 | 3.7106e−02 |
| (“OTOF_2”) | | | | | | | | |
| CA10 | chr17 | 47591160 | 47593160 | 0.02381 | 0.459416 | 0.38194 | −4.3561e−01 | 6.8395e−03 |
| RASGRP4 | chr19 | 43607785 | 43609785 | 0 | 0.44898 | 0.45238 | −4.4898e−01 | 6.8079e−05 |
| CES1 | chr16 | 54423576 | 54425576 | 0.567409 | 0.950770 | 0.874510 | −0.38336 | 9.4458e−39 |
| ALX1 | chr12 | 84197166 | 84199166 | 0.014286 | 0.468994 | 0.0803 | −4.5471e−01 | 2.3810e−02 |
| C11orf48 | chr11 | 62194701 | 62196701 | 0.185185 | 0.642125 | 0.17698 | −4.5694e−01 | 2.7591e−09 |
| LYRM7 | chr5 | 130533539 | 130535539 | 0 | 0.458333 | 0.08889 | −4.5833e−01 | 1.0973e−02 |
| MGAT5B | chr17 | 72379323 | 72381323 | 0.238272 | 0.70303 | 0.38492 | −4.6476e−01 | 3.7228e−05 |
| MKLN1 | chr7 | 130444394 | 130446394 | 0 | 0.474074 | 0.02857 | −4.7407e−01 | 6.6330e−03 |
| RTDR1 | chr22 | 21813241 | 21815241 | 0.022222 | 0.49881 | 0.21429 | −4.7659e−01 | 3.4903e−05 |
| STXBP4 | chr17 | 50400053 | 50402053 | 0.082532 | 0.563946 | 0.10606 | −4.8141e−01 | 2.3516e−07 |
| COX11 | chr17 | 50400124 | 50402124 | 0.082532 | 0.563946 | 0.10606 | −4.8141e−01 | 2.3516e−07 |
| C11orf83 | chr11 | 62194817 | 62196817 | 0.059829 | 0.557919 | 0.0653 | −4.9809e−01 | 8.0058e−06 |
| SNCA | chr4 | 90977470 | 90979470 | 0 | 0.5 | 0.03571 | −5.0000e−01 | 2.4462e−02 |
| POLD4 | chr11 | 66876593 | 66878593 | 0.029412 | 0.543651 | 0.08012 | −5.1424e−01 | 4.3830e−03 |
| CLNS1A | chr11 | 77025499 | 77027499 | 0.033147 | 0.559829 | 0.06905 | −5.2668e−01 | 1.3793e−03 |
| DNAJC19 | chr3 | 182189224 | 182191224 | 0.046875 | 0.577778 | 0 | −5.3090e−01 | 2.6448e−02 |
| JAM2 | chr21 | 25932459 | 25934459 | 0 | 0.5375 | 0.07152 | −5.3750e−01 | 4.2675e−03 |
| ITGA4 | chr2 | 182028863 | 182030863 | 0.013889 | 0.555556 | 0.04959 | −5.4167e−01 | 4.8440e−04 |
| DGUOK | chr2 | 74006460 | 74008460 | 0.018519 | 0.578283 | 0.51968 | −5.5976e−01 | 1.0321e−05 |
| LOC387647 | chr10 | 29737506 | 29739506 | 0.007937 | 0.571429 | 0.02381 | −5.6349e−01 | 6.2843e−05 |
| ABCA5 | chr17 | 64833918 | 64835918 | 0.03 | 0.64 | 0.01818 | −6.1000e−01 | 4.7083e−05 |
| NUBPL | chr14 | 31099341 | 31101341 | 0 | 0.620833 | 0.08333 | −6.2083e−01 | 4.3942e−02 |
| SNRPF | chr12 | 94775839 | 94777839 | 0.041667 | 0.666667 | 0.425 | −6.2500e−01 | 3.2710e−03 |
| CA10 | chr17 | 47591376 | 47593376 | 0.055556 | 0.721939 | 0.14583 | −6.6638e−01 | 3.5299e−04 |
| PSCA | chr8 | 143757876 | 143759876 | 0.02381 | 0.75 | 0.16667 | −7.2619e−01 | 1.7683e−04 |
| BC035336 | chr18 | 72368599 | 72370599 | 0.011905 | 0.75 | 0.29464 | −7.3810e−01 | 7.7401e−04 |
| DDX11 | chr12 | 9491035 | 9493035 | 0 | 0.75 | 0 | −7.5000e−01 | 1.5648e−03 |
| KCNIP4 | chr5 | 169712458 | 169714458 | 0.0384615 | 0.8083333 | 0.038462 | −7.6987e−01 | 2.0176e−04 |
| PDIA3p | chr1 | 145115053 | 145117053 | 0.2253535 | 1 | 0 | −7.7465e−01 | 3.9184e−08 |
| POM121 | chr7 | 71986871 | 71988871 | 0.042361 | 1 | 0 | −9.5764e−01 | 1.2626e−03 |
| INGX | chrX | 70628329 | 70630329 | 0.961051 | 0.182601 | 0.270981 | 0.77845 | 1.3127e−218 |
| NLGN2 | chr17 | 7251225 | 7253225 | 0.177045 | 0.779733 | 0.599882 | −0.60269 | 1.3455e−75 |

| | | mDifference | | |
|---|---|---|---|---|
| gene ID | Class_C_vs_A | C_vs_J | pValue_C_vs_J | Class_C_vs_J |
| NGDN | Strongly | 2.5672e−01 | 7.6332e−04 | Hypo_methylated |
| | Hypometh | | | |
| HAL | Strongly | 1.2645e−01 | 3.7791e−01 | insignificant |
| | Hypometh | | | |
| S100A1 | Strongly | 3.2639e−01 | 1.4681e−01 | insignificant |
| | Hypometh | | | |
| OTOF | Strongly | 9.1027e−02 | 4.2993e−01 | insignificant |
| (“OTOF_1”) | Hypometh | | | |
| MTBP | Strongly | 3.7500e−01 | 2.4611e−02 | Strongly |
| | Hypometh | | | Hypometh |
| MRPL13 | Strongly | 3.7500e−01 | 2.4611e−02 | Strongly |
| | Hypometh | | | Hypometh |
| HOXB6 | Strongly | 5.5152e−01 | 2.8749e−04 | Strongly |
| | Hypometh | | | Hypometh |
| KAZN | Strongly | 1.4848e−01 | 1.6444e−01 | insignificant |
| | Hypometh | | | |
| MORC2- | Strongly | 9.4762e−02 | 1.4695e−02 | Hypo_methylated |
| AS1 | Hypometh | | | |
| LCK | Strongly | −3.5219e−02 | 5.1970e−02 | insignificant |
| | Hypometh | | | |
| ZNF248 | Strongly | 3.8858e−01 | 2.7050e−01 | insignificant |
| | Hypometh | | | |
| ANAPC7 | Strongly | 3.8355e−01 | 1.6563e−03 | Strongly |
| | Hypometh | | | Hypometh |
| SEC1 | Strongly | 4.4128e−01 | 3.2106e−02 | Strongly |
| | Hypometh | | | Hypometh |
| THOC6 | Strongly | 4.6429e−01 | 1.3471e−02 | Strongly |
| | Hypometh | | | Hypometh |
| HCFC1R1 | Strongly | 4.6429e−01 | 1.3471e−02 | Strongly |
| | Hypometh | | | Hypometh |
| ABCE1 | Strongly | 3.8818e−01 | 6.7088e−02 | insignificant |
| | Hypometh | | | |
| ANAPC10 | Strongly | 3.8818e−01 | 6.7088e−02 | insignificant |
| | Hypometh | | | |
| DDX18 | Strongly | 4.6939e−01 | 1.6118e−03 | Strongly |
| | Hypometh | | | Hypometh |
| CD1D | Strongly | 6.2500e−02 | 2.3825e−02 | Hypo_methylated |
| | Hypometh | | | |
| ALKBH6 | Strongly | 6.0417e−01 | 7.0603e−03 | Strongly |
| | Hypometh | | | Hypometh |
| S100A13 | Strongly | 4.9497e−01 | 7.7479e−02 | insignificant |
| | Hypometh | | | |
| SPIN3 | Strongly | 0.0000e+00 | 1.0000e+00 | insignificant |
| | Hypermeth | | | |
| LALBA | Strongly | −8.5000e−01 | 4.3290e−02 | Strongly |
| | Hypermeth | | | Hypermeth |
| EIF4A1 | Strongly | −8.1685e−02 | 7.7771e−01 | insignificant |
| | Hypermeth | | | |
| CENPF | Strongly | 3.8282e−03 | 1.0000e+00 | insignificant |
| | Hypermeth | | | |
| AIRE | Strongly | 7.5488e−02 | 1.6941e−01 | insignificant |
| | Hypermeth | | | |
| ANKRD13C | Strongly | 7.7247e−03 | 2.5622e−01 | insignificant |
| | Hypermeth | | | |
| GHSR | Strongly | −2.2865e−02 | 4.6504e−01 | insignificant |
| | Hypermeth | | | |
| EIF3G | Strongly | −2.1429e−02 | 1.0000e+00 | insignificant |
| | Hypermeth | | | |
| NOLC1 | Strongly | −1.0684e−02 | 1.0000e+00 | insignificant |
| | Hypermeth | | | |
| TPX2 | Strongly | −1.6164e−01 | 1.0000e+00 | insignificant |
| | Hypermeth | | | |
| BDNF | Strongly | −1.6583e−01 | 1.0273e−01 | insignificant |
| | Hypermeth | | | |
| FAM149B1 | Strongly | 2.5794e−03 | 1.0000e+00 | insignificant |
| | Hypermeth | | | |
| ECD | Strongly | 2.5794e−03 | 1.0000e+00 | insignificant |
| | Hypermeth | | | |
| OGG1 | Strongly | −4.6951e−02 | 1.0000e+00 | insignificant |
| | Hypermeth | | | |
| KATNAL1 | Strongly | −1.4744e−01 | 2.7143e−02 | Hyper_methylated |
| | Hypermeth | | | |
| IGFBP5 | Strongly | −1.9471e−01 | 1.9602e−01 | insignificant |
| | Hypermeth | | | |
| MRPS15 | Strongly | 2.4640e−02 | 3.3990e−02 | Hypo_methylated |
| | Hypermeth | | | |
| HIST1H4K | Strongly | −3.3303e−01 | 3.9395e−01 | insignificant |
| | Hypermeth | | | |
| CMYA5 | Strongly | −2.7869e−01 | 1.5228e−08 | Hyper_methylated |
| | Hypermeth | | | |
| ARMC1 | Strongly | 2.4793e−03 | 1.0000e+00 | Insignificant |
| | Hypermeth | | | |
| LOC286002 | Strongly | −3.9385e−02 | 1.0000e+00 | insignificant |
| | Hypermeth | | | |
| PAIP2 | Strongly | 0.0000e+00 | 1.0000e+00 | insignificant |
| | Hypermeth | | | |
| MARCKS | Strongly | −1.7812e−01 | 4.0535e−01 | insignificant |
| | Hypermeth | | | |
| DNAJC25- | Strongly | −1.7783e−02 | 3.3339e−01 | insignificant |
| GNG10 | Hypermeth | | | |
| BLZF1 | Strongly | −1.4286e−01 | 1.0000e+00 | insignificant |
| | Hypermeth | | | |
| NME7 | Strongly | −1.4286e−01 | 1.0000e+00 | insignificant |
| | Hypermeth | | | |
| C12orf26 | Strongly | −1.1781e−01 | 1.0000e+00 | insignificant |
| | Hypermeth | | | |
| CCDC59 | Strongly | −1.1781e−01 | 1.0000e+00 | insignificant |
| | Hypermeth | | | |
| MCRS1 | Strongly | −1.9160e−02 | 1.0515e−01 | insignificant |
| | Hypermeth | | | |
| RBM4B | Strongly | −1.9907e−01 | 2.4329e−01 | insignificant |
| | Hypermeth | | | |
| MIR548H4 | Strongly | −2.6987e−01 | 5.0832e−01 | insignificant |
| | Hypermeth | | | |
| TEKT1 | Strongly | −2.2078e−02 | 1.0000e+00 | insignificant |
| | Hypermeth | | | |
| TMEM184C | Strongly | −6.6215e−02 | 3.8217e−01 | insignificant |
| | Hypermeth | | | |
| TTC22 | Strongly | 8.5971e−02 | 7.2366e−02 | insignificant |
| | Hypermeth | | | |
| FAM164A | Strongly | 1.7890e−02 | 3.7131e−01 | insignificant |
| | Hypermeth | | | |
| PACRGL | Strongly | −3.7879e−02 | 1.9590e−02 | Hyper_methylated |
| | Hypermeth | | | |
| CCDC58 | Strongly | −8.8141e−02 | 5.8540e−01 | insignificant |
| | Hypermeth | | | |
| TMEM126B | Strongly | −6.9088e−02 | 4.6535e−01 | insignificant |
| | Hypermeth | | | |
| BLCAP | Strongly | −1.0118e−01 | 1.2019e−01 | insignificant |
| | Hypermeth | | | |
| LOC100131564 | Strongly | −7.1385e−03 | 1.0000e+00 | insignificant |
| | Hypermeth | | | |
| GTF3C2 | Strongly | −4.3246e−02 | 7.3129e−01 | insignificant |
| | Hypermeth | | | |
| RBP5 | Strongly | −2.0833e−01 | 5.6415e−02 | insignificant |
| | Hypermeth | | | |
| NVL | Strongly | 1.7857e−02 | 1.0000e+00 | insignificant |
| | Hypermeth | | | |
| NARG2 | Strongly | −1.7857e−02 | 4.8718e−01 | insignificant |
| | Hypermeth | | | |
| PTPLAD2 | Strongly | 5.0758e−02 | 8.6006e−01 | insignificant |
| | Hypermeth | | | |
| OTOF | Strongly | −4.2408e−01 | 7.2470e−03 | Strongly |
| (“OTOF_2”) | Hypermeth | | | Hypermeth |
| CA10 | Strongly | −3.5813e−01 | 6.4840e−02 | insignificant |
| | Hypermeth | | | |
| RASGRP4 | Strongly | −4.5238e−01 | 4.9493e−04 | Strongly |
| | Hypermeth | | | Hypermeth |
| CES1 | Strongly | −0.3071 | 3.6017e−12 | Hypermethylated |
| | Hypermeth | | | |
| ALX1 | Strongly | −6.6017e−02 | 8.3539e−02 | insignificant |
| | Hypermeth | | | |
| C11orf48 | Strongly | 8.2032e−03 | 9.0947e−02 | insignificant |
| | Hypermeth | | | |
| LYRM7 | Strongly | −8.8889e−02 | 4.9277e−01 | insignificant |
| | Hypermeth | | | |
| MGAT5B | Strongly | −1.4665e−01 | 5.7569e−03 | Hyper_methylated |
| | Hypermeth | | | |
| MKLN1 | Strongly | −2.8571e−02 | 4.1463e−01 | insignificant |
| | Hypermeth | | | |
| RTDR1 | Strongly | −1.9206e−01 | 3.5803e−01 | insignificant |
| | Hypermeth | | | |
| STXBP4 | Strongly | −2.3529e−02 | 4.8082e−01 | insignificant |
| | Hypermeth | | | |
| COX11 | Strongly | −2.3529e−02 | 4.8082e−01 | insignificant |
| | Hypermeth | | | |
| C11orf83 | Strongly | −5.4701e−03 | 5.1976e−01 | insignificant |
| | Hypermeth | | | |
| SNCA | Strongly | −3.5714e−02 | 5.1531e−01 | insignificant |
| | Hypermeth | | | |
| POLD4 | Strongly | −5.0707e−02 | 2.6567e−01 | insignificant |
| | Hypermeth | | | |
| CLNS1A | Strongly | −3.5901e−02 | 1.0000e+00 | insignificant |
| | Hypermeth | | | |
| DNAJC19 | Strongly | 4.6875e−02 | 1.0034e−01 | insignificant |
| | Hypermeth | | | |
| JAM2 | Strongly | −7.1515e−02 | 3.1602e−01 | insignificant |
| | Hypermeth | | | |
| ITGA4 | Strongly | −3.5698e−02 | 1.0000e+00 | insignificant |
| | Hypermeth | | | |
| DGUOK | Strongly | −5.0116e−01 | 1.0093e−03 | Strongly |
| | Hypermeth | | | Hypermeth |
| LOC387647 | Strongly | −1.5873e−02 | 1.0000e+00 | insignificant |
| | Hypermeth | | | |
| ABCA5 | Strongly | 1.1818e−02 | 6.5853e−01 | insignificant |
| | Hypermeth | | | |
| NUBPL | Strongly | −8.3333e−02 | 1.0000e+00 | insignificant |
| | Hypermeth | | | |
| SNRPF | Strongly | −3.8333e−01 | 3.3669e−01 | insignificant |
| | Hypermeth | | | |
| CA10 | Strongly | −9.0278e−02 | 2.8875e−01 | insignificant |
| | Hypermeth | | | |
| PSCA | Strongly | −1.4286e−01 | 1.4757e−01 | insignificant |
| | Hypermeth | | | |
| BC035336 | Strongly | −2.8274e−01 | 1.5340e−01 | insignificant |
| | Hypermeth | | | |
| DDX11 | Strongly | 0.0000e+00 | 1.0000e+00 | insignificant |
| | Hypermeth | | | |
| KCNIP4 | Strongly | 0.0000e+00 | 1.0000e+00 | insignificant |
| | Hypermeth | | | |
| PDIA3p | Strongly | 2.2535e−01 | 5.5226e−02 | insignificant |
| | Hypermeth | | | |
| POM121 | Strongly | 4.2361e−02 | 1.0000e+00 | insignificant |
| | Hypermeth | | | |
| INGX | Strongly | 0.69007 | 3.3692e−176 | Strongly |
| | Hypometh | | | Hypometh |
| NLGN2 | Strongly | −0.42284 | 2.0353e−31 | Strongly |
| | Hypermeth | | | Hypermeth |

“C”—indicates control;
“A” indicates autism sample A;
“J” indicates autism sample
J. mRatio—indicates methylation ratio;
mDifference - indicate mRatio difference.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,786,146
U.S. Patent Publn. 2010/0304992
U.S. Patent Publn. 2011/0046009
Adorjan et al., Nucleic Acids Res., 30(5):e21, 2002.
Akey et al., Genomics, 80(4):376-84, 2002.
Bailey et al., Psychol. Med., 25:63-77, 1995.
Bianco et al., Hum. Mutat., 14(4):289-93, 1999.
Callinan, Hum. Mol. Genet., 15:R95-101, 2006.
Colella et al., BioTechniques, 35(1):146-50, 2003.
Cook et al., Curr. Opin. Pediatr., 8:348-354, 1996.
Eads et al., Nucleic Acids Res., 28(8):E32, 2000.
Ehrich et al. Proc. Natl. Acad. Sci. USA, 102(44):15785-90, 2005.
El-Maarri, Adv. Exp. Med. Biol., 544:197-204, 2003.
Folstein et al., Nat. Rev. Genet., 2:943-955, 2001.
Fraga, BioTechniques, 33(3):632, 634, 636-49, 2002.
Frommer et al. Proc. Natl. Acad. Sci. USA, 89(5):1827-31, 1992.
Gonzalgo and Jones, Nucleic Acids Res., 25(12):2529-31, 1997.
Hollander et al., Neuropsychopharmacology, 30:582-589, 2005.
Keshet et al., Nat. Genet., 38(2):149-53, 2006.
Kristensen et al., Nucleic Acids Res., 36(7):e42, 2008.
Laird, Nat. Rev. Cancer, 3(4):253-66, 2003.
Matin et al., Hum. Mutat., 20(4):305-11, 2002.
PCT Pub. No. WO/2010/114821
PCT Pub. No. WO/2011/109529
Rand et al., Methods, 27(2):114-20, 2002.
Tost et al. BioTechniques, 35(1):152-6, 2003.
Uhlmann et al., Electrophoresis, 23(24):4072-9, 2002.
Weber et al., Nat. Genet., 37(8):853-62, 2005.
Wojdacz and Dobrovic, Nucleic Acids Res., 35(6):e41, 2007.
Wong et al. BioTechniques, 41(6):734-9, 2006.
Xiong et al., Nucleic Acids Res., 25(12):2532-2534, 1997.

1. A method of analysis comprising determining a methylation status in two or more genes in a patient sample from a patient suspected of having an autism spectrum disorder, the genes selected from the group consisting of CES1, NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121.

2. The method of claim 1, wherein the two or more genes is selected from the group consisting of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, POM121 and NLGN2.

3. The method of claim 2, wherein the two or more genes is selected from the group consisting of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, POM121 and NLGN2.

4. The method of claim 3, wherein the two or more genes is selected from the group consisting of SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, RBP5, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, NLGN2 and POM121.

5. The method of claim 1, wherein the two or more genes is selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13 and INGX.

6. The method of claim 5, wherein the two or more genes is selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13 and INGX.

7. The method of claim 6, wherein the two or more genes is selected from the group consisting of NGDN, HAL, S100A1, OTOF_{—}1, MTBP, MRPL13, HOXB6, KAZN, MORC2, LCK, ZNF248, ANAPC7, SEC1, THOC6, HCFC1R1, ABCE1, ANAPC10, DDX18, CD1D, ALKBH6, S100A13 and INGX.

8. The method of claim 1, comprising determining a methylation status of the INGX, NLGN2 and CES1 genes.

9. (canceled)

10. The method of claim 1, wherein said determining comprises determining a methylation status in 3, 4, 5, 6, 7, 8, 9, 10 or more of said genes.

11. (canceled)

12. The method of claim 1 wherein the patient is a human.

13. The method of claim 1, wherein the sample is a blood sample.

14. (canceled)

15. The method of claim 1, wherein the sample is obtained from a third party.

16. The method of claim 1, wherein determining a methylation status comprises determining the nucleotide positions in the gene that comprise methylation.

17. The method of claim 1, wherein determining a methylation status comprises determining the proportion of methylation at a nucleotide positions in the gene.

18. The method of claim 1, wherein determining a methylation status comprises determining the proportion of nucleotide positions that are methylated in the gene sequence.

19. The method of claim 1, wherein determining a methylation status comprises comparing the proportion of methylation in the genes to a the reference level.

20. The method of claim 19, wherein the reference level is from a sample of a patient know to have an autism spectrum disorder or know to not have an autism spectrum disorder.

21. The method of claim 1, wherein determining a methylation status comprises performing a method selected from the group consisting of methylation specific PCR (MSP), real-time methylation specific PCR, methylation-sensitive single-strand conformation analysis (MS-SSCA), quantitative methylation specific PCR (QMSP), PCR using a methylated DNA-specific binding protein, high resolution melting analysis (HRM), methylation-sensitive single-nucleotide primer extension (MS-SnuPE), base-specific cleavage/MALDI-TOF, PCR, real-time PCR, Combined Bisulfite Restriction Analysis (COBRA), methylated DNA immunoprecipitation (MeDIP), a microarray-based method, pyrosequencing, and bisulfite sequencing.

22. (canceled)

23. The method of claim 1, wherein determining a methylation status comprises treating the sample with bisulfate.

24. (canceled)

25. A method for treating a patient having an autism spectrum disorder or at risk for having an autism spectrum disorder comprising administering a therapy to the patient, wherein the patient was previously determined to have an increased or decreased level of methylation in two or more of the genes selected from the group consisting of NGDN, HAL, S100A1, OTOF_{—}1, MTBP, MRPL13, HOXB6, KAZN, MORC2, LCK, ZNF248, ANAPC7, SECT, THOC6, HCFC1R1, ABCE1, ANAPC10, DDX18, CD1D, ALKBH6, S100A13, SPIN3, LALBA, EIF4A1, CENPF, AIRE, ANKRD13C, GHSR, EIF3G, NOLC1, TPX2, BDNF, FAM149B1, ECD, OGG1, KATNAL1, IGFBP5, MRPS15, HIST1H4K, CMYA5, ARMC1, LOC286002, PAIP2, MARCKS, DNAJC25, BLZF1, NME7, C12orf26, CCDC59, MCRS1, RBM4B, MIR548H4, TEKT1, TMEM184C, TTC22, FAM164A, PACRGL, CCDC58, TMEM126B, BLCAP, LOC100131564, GTF3C2, REPS, NVL, NARG2, PTPLAD2, OTOF_{—}2, CA10, RASGRP4, CES1, ALX1, C11orf48, LYRM7, MGAT5B, MKLN1, RTDR1, STXBP4, COX11, C11orf83, SNCA, POLD4, CLNS1A, DNAJC19, JAM2, ITGA4, DGUOK, LOC387647, ABCA5, NUBPL, SNRPF, CA10, PSCA, BC035336, DDX11, KCNIP4, PDIA3p, INGX, NLGN2 and POM121 relative to a reference level.

26-38. (canceled)
